(19) United States
(12) Patent Application Publication
(10) Pub. No.: US 2025/0044119 A1
(43) Pub. Date: Feb. 6, 2025

(54) UNIQUE HIKING INTERACTION VIA IOT DATA ASSIMILATION

(71) Applicant: International Business Machines Corporation, Armonk, NY (US)

(72) Inventors: Jeremy R. Fox, Georgetown, TX (US); Alexander Reznicek, Troy, NY (US); Martin G. Keen, Cary, NC (US); Bahman Hekmatshoartabari, White Plains, NY (US)

(21) Appl. No.: 18/363,780

(22) Filed: Aug. 2, 2023

(51) Int. Cl. G01C 21/00; G16Y 20/10; G16Y 40/50; G16Y 40/60

(52) U.S. Cl. CPC G01C 21/3826; G01C 21/3811; G16Y 20/10; G16Y 40/50; G16Y 40/60

(57) ABSTRACT

The present inventive concept provides for a method of unique hiking interaction via IoT data assimilation. The method includes obtaining health data for a user and location data for a location that includes at least one activity trail. Health features are extracted from the health data and terrain features are extracted from the location data. The extracted health features and the extracted terrain features are analysed and mapped. The extracted health features include biometric measurements from at least one IoT device and the extracted terrain features include characteristics of the at least one activity trail. A unique activity experience is calculated for the user to perform a preselected activity on the at least one activity trail based at least in part on the analysed and mapped extracted health features and the extracted terrain features.

### BACKGROUND

Exemplary embodiments of the present inventive concept relate to unique hiking interaction, and more particularly, to unique hiking interaction via IoT data assimilation.

Participation in outdoor activities (e.g., hiking, mountain biking, rock climbing, etc.) has steadily increased over the last several decades with a particularly pronounced increase observed in the last several years. While outdoor activities offer a healthful and inexpensive means of exercising, trails for outdoor activities can present potential challenges and even dangers to an unwary, novice, unfamiliar, and/or overconfident participant. The CDC reports hiking as the third most common source of injury in the outdoors, accounting for thousands of deaths annually. Participants can get lost, experience a medical emergency, or require help in some other capacity. Trail applications exist that can provide participants with trail maps, user locations thereon, estimated completion times, and difficulty levels. However, current trail applications only consider the average participant and average completion times-nothing specific to the individual participant or instant circumstances (e.g., a hike in summer versus winter). The subjective difficulty of a given trail and an estimated completion time thereof can vary widely depending on a multitude of factors, such as a user fitness level, selected route, equipment used, relevant activity experience, trail conditions, time of day, weather, etc. Crowd sourced user comments regarding trail conditions, when available, are frequently outdated and no longer relevant. In addition, participants frequently overlook or inaccurately evaluate potential safety concerns before embarking on a trail, such as poor cellular reception in the vicinity, insufficient phone battery for entirety of activity, dangerous local wildlife, projected participant position on the trail at sunset, etc.

### SUMMARY

Exemplary embodiments of the present inventive concept relate to a method, a computer program product, and a system for unique hiking interaction via IoT data assimilation.

According to an exemplary embodiment of the present inventive concept, a method of unique hiking interaction via IoT data assimilation is provided. The method includes obtaining health data for a user and location data for a location that includes at least one activity trail. Health features are extracted from the health data and terrain features are extracted from the location data. The extracted health features and the extracted terrain features are analysed and mapped. The extracted health features include biometric measurements from at least one IoT device and the extracted terrain features include characteristics of the at least one activity trail. A unique activity experience is calculated for the user to perform a preselected activity on the at least one activity trail based at least in part on the analysed and mapped extracted health features and the extracted terrain features.

According to an exemplary embodiment of the present inventive concept, a computer program product is provided. The computer program product includes one or more computer-readable storage media and program instructions stored on the one or more non-transitory computer-readable storage media capable of performing a method. The present inventive concept provides for a method of unique hiking interaction via IoT data assimilation. The method includes obtaining health data for a user and location data for a location that includes at least one activity trail. Health features are extracted from the health data and terrain features are extracted from the location data. The extracted health features and the extracted terrain features are analysed and mapped. The extracted health features include biometric measurements from at least one IoT device and the extracted terrain features include characteristics of the at least one activity trail. A unique activity experience is calculated for the user to perform a preselected activity on the at least one activity trail based at least in part on the analysed and mapped extracted health features and the extracted terrain features.

According to an exemplary embodiment of the present inventive concept, a computer system is provided. The computer system includes one or more computer processors, one or more computer-readable storage media, and program instructions stored on the one or more of the computer-readable storage media for execution by at least one of the one or more processors capable of performing a method of unique hiking interaction via IoT data assimilation. The method includes obtaining health data for a user and location data for a location that includes at least one activity trail. Health features are extracted from the health data and terrain features are extracted from the location data. The extracted health features and the extracted terrain features are analysed and mapped. The extracted health features include biometric measurements from at least one IoT device and the extracted terrain features include characteristics of the at least one activity trail. A unique activity experience is calculated for the user to perform a preselected activity on the at least one activity trail based at least in part on the analysed and mapped extracted health features and the extracted terrain features.

### BRIEF DESCRIPTION OF THE DRAWINGS

The following detailed description, given by way of example and not intended to limit the exemplary embodiments solely thereto, will best be appreciated in conjunction with the accompanying drawings, in which:

FIG. 1 illustrates a schematic diagram of computing environment 100 including a unique hiking interaction via IoT data assimilation program 150, in accordance with an exemplary embodiment of the present inventive concept.

FIG. 2 illustrates a block diagram of components included in the unique hiking interaction via IoT data assimilation program 150, in accordance with an exemplary embodiment of the present inventive concept.

FIG. 3 illustrates a flowchart of a method of unique hiking interaction via IoT data assimilation 300, in accordance with an exemplary embodiment of the present inventive concept.

It is to be understood that the included drawings are not necessarily drawn to scale/proportion. The included drawings are merely schematic examples to assist in understanding of the present inventive concept and are not intended to portray fixed parameters. In the drawings, like numbering may represent like elements.

### DETAILED DESCRIPTION

Exemplary embodiments of the present inventive concept are disclosed hereafter. However, it shall be understood that the scope of the present inventive concept is dictated by the claims. The disclosed exemplary embodiments are merely illustrative of the claimed system, method, and computer program product. The present inventive concept may be embodied in many different forms and should not be construed as limited to only the exemplary embodiments set forth herein. Rather, these included exemplary embodiments are provided for completeness of disclosure and to facilitate an understanding to those skilled in the art. In the detailed description, discussion of well-known features and techniques may be omitted to avoid unnecessarily obscuring the presented exemplary embodiments.

References in the specification to “one embodiment,” “an embodiment,” “an exemplary embodiment,” etc., indicate that the embodiment described may include a particular feature, structure, or characteristic, but not every embodiment may necessarily include that feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether explicitly described.

In the interest of not obscuring the presentation of the exemplary embodiments of the present inventive concept, in the following detailed description, some processing steps or operations that are known in the art may have been combined for presentation and for illustration purposes, and in some instances, may have not been described in detail. Additionally, some processing steps or operations that are known in the art may not be described at all. The following detailed description is focused on the distinctive features or elements of the present inventive concept according to various exemplary embodiments.

The present inventive concept provides for a method, system, and computer program product for unique hiking interaction via IoT data assimilation. The present inventive concept can determine a user fitness level overall and/or for a given activity (e.g., personalized hiking capability) based on prior IoT data (e.g., biometrics and/or collected fitness tracker data) and can provide a user interface with appropriately matched activity trails (e.g., hiking trails) and/or routes to undertake which are appropriately matched to their user fitness level. Explanations and personalized completion time estimates for the matched activity trails can be provided to the user. The present inventive concept can also provide personalized warnings to the user, such as overambitious hikes and potentially unforeseen circumstances (e.g., return after sunset, significantly more elevation gain than the user has experienced before, river crossings during rainy season, etc.). The present inventive concept can also reduce the amount of time needed to search for a lost/injured user on an activity trail because rescue teams will be able to concentrate on a last known position and/or a projected position indicated by last known pace and/or biometrics of the user.

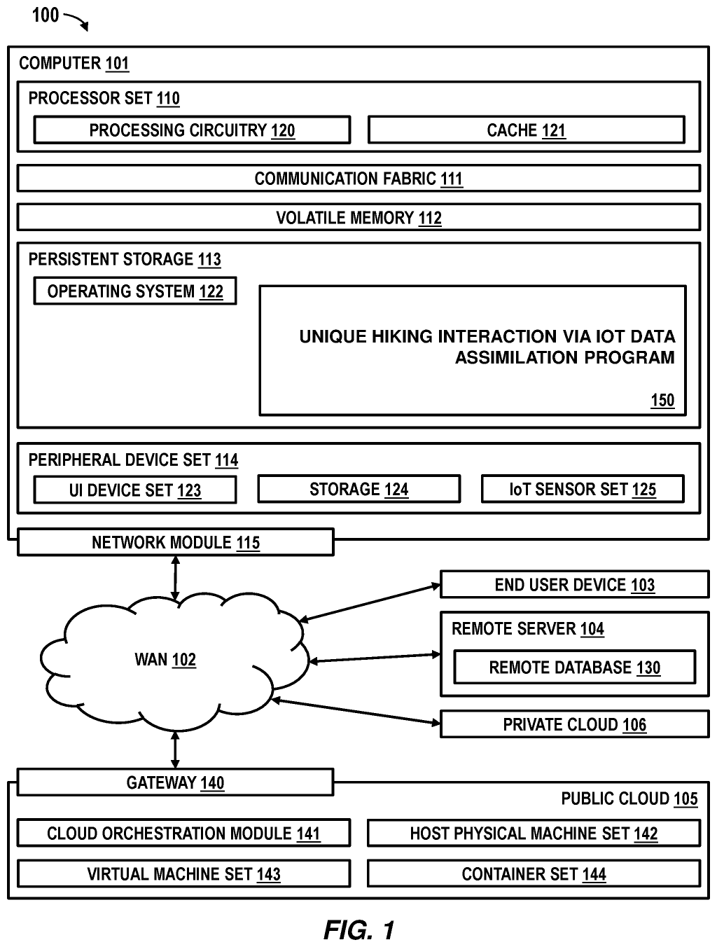

FIG. 1 illustrates a schematic diagram of computing environment 100 including the unique hiking interaction via IoT data assimilation program 150, in accordance with an exemplary embodiment of the present inventive concept.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as the unique hiking interaction via IoT data assimilation program 150. In addition to block 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 150 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

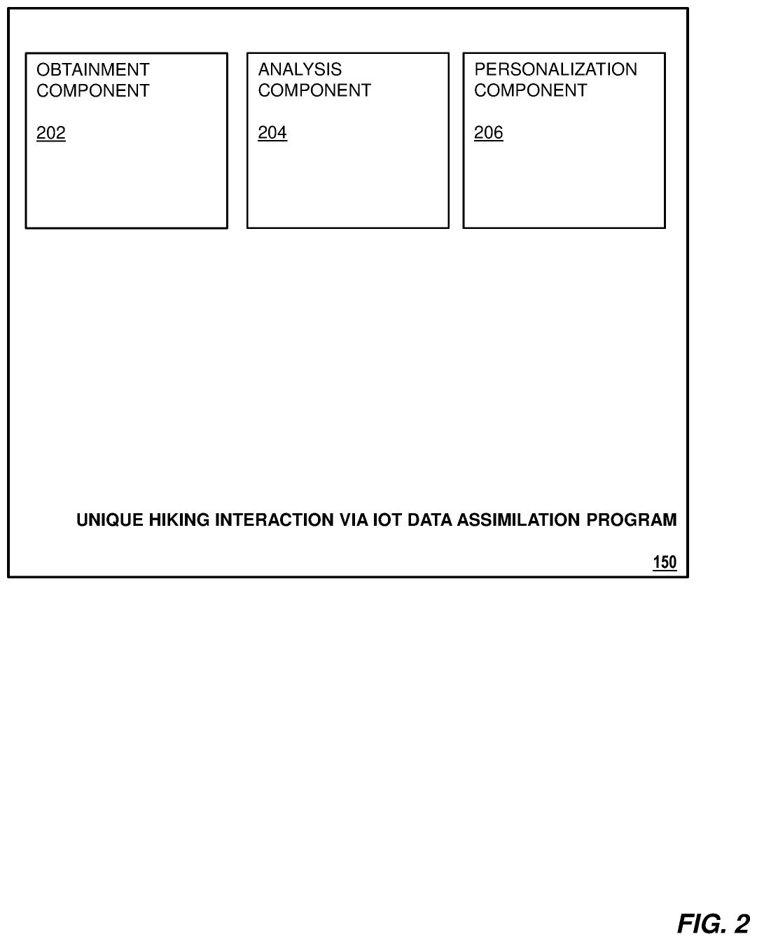

FIG. 2 illustrates a block diagram of components included in the unique hiking interaction via IoT data assimilation program 150, in accordance with an exemplary embodiment of the present inventive concept.

The obtainment component 202 can obtain health data for at least one user. The health data can include biometric data from relevant repository information, at least one user IoT device (e.g., smartphone, smartwatch, smartscale, fitness tracker, etc.), authorized electronic health records, user inputs (e.g., preexisting conditions, scheduled activity time, pain scores, symptoms, equipment used, desired activity difficulty, fitness goal, activity experience level, etc.), etc. The obtainment component 202 can extract health features from the obtained health data, such as user height, weight, age, hydration levels, arrythmias, oxygen levels, body temperatures, crashes/falls, resting heart rates, activity cadences/paces/completions, body compositions, VO₂ maxes, sleep quality measurements, heart rate variabilities, gait stability measurements, motion tracking, calories consumed/expended, health conditions/prosthetics/implants/injuries, medical considerations/limitations/proscriptions, durations/times/frequencies of activity/rest/inactivity, activity types (e.g., mountain biking, hiking, rock climbing, etc.), activity locations, abrupt activity discontinuations/maximum heart rates/predetermined heart rate elevations, total steps, total distances, flights climbed, emergency contacts, etc. The user's personal information (e.g., the extracted health features and/or the extracted terrain features) can be stored in a repository and/or linked to a personalized user account accessible, for example, by a unique activity trail experience interface.

The obtainment component 202 can also obtain location data (e.g., relevant repository data, weather data, IoT data, sunrise/sunset times/orientations/obscurement by topographical features relative to an activity trail, trail application multimedia, streamed/recorded video, satellite imaging, digital maps, user inputs, etc.) for at least one location (e.g., predetermined radius from user home/preselected location, extracted activity location(s), user selected location(s), location(s) automatically selected by a personalization component 206 (described below)). The user's personal activity history can be retrieved from past and/or current health data. The at least one location can include at least one activity trail for performing at least one preselected activity. The obtainment component 202 can extract terrain features from the location data, including activity trails and characteristics thereof (e.g., names, user positions and/or times, summits/landmarks/milestones, activity types, routes, compass directions, altitudes/atmospheric oxygen levels, elevation gain/loss, segments, shapes, inclusion of ladders/ropes/obstacles, estimated/actual times for user completion/discontinuation, equipment used/suggested and relative estimated/actual times for completion/discontinuation, published/reported difficulty levels, GPS coordinates, user reported trail conditions/hazards (e.g., rockfall, mud/dry, downed trees, leafy debris, incomplete/missing activity trail markers, slippery rocks, streams and widths/depths thereof, wildlife, etc.)), topography, wind, weather conditions, reported emergencies/types/severities, cellular service coverage, distance from first responders, etc.

The obtainment component 202 can obtain the health data and/or the location data and extract features therefrom continuously, at predetermined intervals, and/or pursuant to a triggering condition. For example, the obtainment component 202 can obtain/extract features from the location data upon a user position being within a predetermined distance from an activity trail, predetermined change in activity pace/cadence/biometrics, user activity trail selection, and/or based on a preselected activity.

For example, a user opts into the unique hiking interaction using IoT data assimilation program 150. The user inputs include hiking for the preselected activity, slightly ambitious difficulty, cardiovascular improvement goal, a departure window, current hydration levels, current caloric consumption, height, weight, age, body composition, history of knee arthritis/pain scores and arrythmias. The obtainment component 202 obtains health data from the user's fitness tracker, smartscale, and smartwatch and extracts health features relevant to hiking capabilities, including heartrate, arrythmia history, activity trails completed and corresponding total steps/distances/rests/elevation gains/elevation losses, and recency/regularity of hiking and activity more generally. The obtainment component 202 also obtains location data corresponding to hiking trails within a commutable distance of the departure window, and extracts terrain features thereof, such as hiking trails, hiking trail conditions, topography, elevation gain/loss, sunset/sunset time and compass directions, total length, stream crossings, temperatures, precipitation, summits, treelines, cellular service coverage, and proximity to first responders.

The analysis component 204 can map the extracted user health features, user inputs, and/or the extracted terrain features. The analysis component 204 can determine a user fitness level in general and/or by activity type (e.g., the preselected activity). The user fitness level can be based on identified activity capability patterns in the mapped features (e.g., user struggles with high altitudes, activity exceeding a predetermined length/time, ascents of a predetermined quantity/length/grade, activity type, weather and/or trail conditions, relative pace/cadence on ascents/descents/flat segments, influence of lapses in activity on subsequent biometrics, etc.). Logistic regression and/or support vector machines can be used to analyse the extracted health features, such as the user's height, weight, age, and other relevant information and added to the personalized user account accessible, for example, by the repository and/or the unique activity experience interface. For example, the user's physical characteristics can be analyzed to determine their activity type capabilities; linear regression can be used to analyze the user's physical characteristics, such as body mass index, heart rate, and other relevant information to assess the user's current activity capabilities. K-means clustering and/or a decision tree can be used to analyze the user's activity history, such as activity types, performance, trail participation, duration, and/or distance, elevation gain/loss, and other relevant information. A random forest can be utilized to analyse the user's recent activities, exercise regimens, and other relevant information to assess a user fitness level. The analysis component 204 can determine activity trail difficulty levels (e.g., by standard path, point/direction of commencement, route, planned rests, interconnected trails, etc.) and/or interconnections thereof based at least in part on the extracted terrain features, the corresponding extracted user health features, user inputs, prior activity trail performance/similarity, and/or the preselected activity, etc. The activity trail difficulty level can also be based in part on a comparison of the user fitness level and the user's historic activity trail performances to user fitness levels and activity trail performances (e.g., completion times) of prior users on the same and/or similar activity trails performing the same preselected activity.

For example, the analysis component 204 maps the extracted health features, user inputs, and the extracted terrain features. The analysis component 204 analyses the mapped features and determines a user fitness level for the preselected activity of hiking and identified hiking capability patterns (e.g., pain and near maximum heart rate with elevation gains greater than predetermined grades/distances alleviated by descent and/or rest, hiking cessation for greater than a predetermined threshold, difficulty with muddy conditions and leafy debris, etc.). The analysis component 204 also analyses the extracted terrain features and determines a plurality of activity trail difficulties included in the locations. The activity trail difficulties are also based in part on mapped features from prior user participants stored in a repository. The analysis component 204 compares the user's determined hiking fitness level to the determined hiking trail difficulties.

The personalization component 206 can receive a current user location (e.g., via accelerometer, compass, and/or dual-frequency GPS to monitor precise location along an activity trail, etc.) or preselected location. Additionally, or alternatively, the personalization component 206 can automatically select at least one location and/or at least one activity trail therein for the preselected activity based on the comparison of the user fitness level and the activity trail difficulty level, and/or the user inputs (e.g., desired difficulty level, fitness goal, preexisting conditions, etc.). The personalization component 206 can also obtain real-time health data (e.g., heartrate, gait stability, falls/crashes, pace/cadence, etc. during the preselected activity) and real-time location data (e.g., recent weather changes). The personalization component 206 can calculate a unique activity experience (e.g., a personalized estimated time for the activity trail overall and/or segment completion for the user, matched activity trails, suggested routes/directions/turn around points/interconnections, potential activity capability/safety concerns, etc.), such as by using a linear regression algorithm. The personalization component 206 can suggest equipment for the activity (e.g., moisture wicking clothes, heat appropriate clothes, bug repellant, bear spray, footwear, trekking poles, camel backpack, flashlights, tents, rainwear, etc.) based on the extracted health features and/or the extracted terrain features. The personalization component 206 can indicate to the user any suggested equipment's projected influence on overall personalized estimated times for the activity trail completion and convey estimated magnitudes of time thereof. For example, an algorithm such as a Naive Bayes classifier can be employed to identify potential safety risks, such as overambitious hikes, estimated return after sunset, far more elevation gain than the user is accustomed, and/or river crossings.

Via the unique activity experience interface, the personalization component 206 can convey the unique activity experience, factors that can influence the user's estimated personalized completion time, and/or considerations/hazards (e.g., lack of cellular service in difficult/dangerous segments, risk of hypothermia, heat exhaustion, minutes of phone battery charge remaining insufficient for completion and predicted user position, sunset before completion and corresponding user position, elevated temperature relative to average, forecast precipitation, potential flash flooding from intersecting water bodies, low tree line and forecast lightning/thunders, risk due to recent cessation from physical activity, etc.), comparative user fitness level, comparative user fitness levels of prior participants (e.g., list, average, frequencies, reported discrepancies from completion estimations/difficulties/extracted terrain features, etc.), estimated personal difficulty points (e.g., muddy segments, steep ascents, cliffs, rock gardens, etc.), the extracted terrain features, etc.

The personalization component 206 can monitor the user's location in real-time (e.g., using the GPS and Kalman filter algorithm, accelerometer, and/or compass) and benchmarks of progress and update the estimated personalized completion time dynamically. The user's motion/position/pace/cadence/biometrics can be compared (e.g., by linear regression) to the unique activity experience predictions (e.g., the personalized estimated time for completion, route, progress benchmarks/landmarks, pace/cadence/biometrics, etc.). The user can be provided with updated feedback and a dynamic, personalized unique activity interaction trail guide. A decision tree can be employed to generate the personalized trail guide based on the user's estimated times for activity progress and their navigational patterns, etc. The personalization component 206 can survey the user (and adjust a calculated user fitness level, unique activity interaction, and/or identified patterns, etc. accordingly) and/or provide feedback to the user based on their relative performance during and/or after the preselected activity. The present inventive concept can send periodic updates to a server, and the user can check out after finishing the hike, like trail logs at trail heads.

For example, the personalization component 206 selects a custom route of interconnected hiking trails for the user within the 1-hour commuting location circumference from their home based on the compared user fitness level and hiking trail difficulties, and the user input of a slightly ambitious hike difficulty with the goal of cardiovascular improvement. The personalization component 206 calculates a unique hiking experience for the user, including an estimated completion time, estimated progress across segments, estimated pace/cadence, and landmarks. The personalization component 206 displays factors via a user interface contributing to the slightly ambitious designation, such as segments with ascents and/or distances slightly above the predetermined thresholds prior to the summit, but with a scheduled rest of 10 minutes at the summit followed by a long descent segment. The personalization component 206 also displays considerations/hazards via the interface such as early effective sunset within a ½ hour of estimated completion time (due to obscurement by the summit), nocturnal predators, sporadic cellular service, significant distance to first responders, low treeline at summit despite forecast thunder, elevated temperature relative to average, excess caloric expenditure versus consumption, and low hydration levels. The personalization component 206 suggests equipment to the user, such as trekking poles, hiking shoes, a camel backpack, and a flashlight with projected impacts on overall completion time and difficulty. The personalization component 206 monitors the user's progress on the trail and obtains real-time weather and real-time biometrics. The personalization component 206 determines that the user's positional heartrate is well above estimations with possible arrythmias just before the slightly ambitious ascent. The user is already 45 minutes below estimated pace, ensuring sunset during a segment including the steep descent. The personalization component 206 modifies the unique hiking interaction and advises the user to take a flat, easy shortcut back estimated to take 30 minutes at present pace and puts first responders on notice (when cellular service becomes momentarily available) in case the user does not check-out within a predetermined time window.

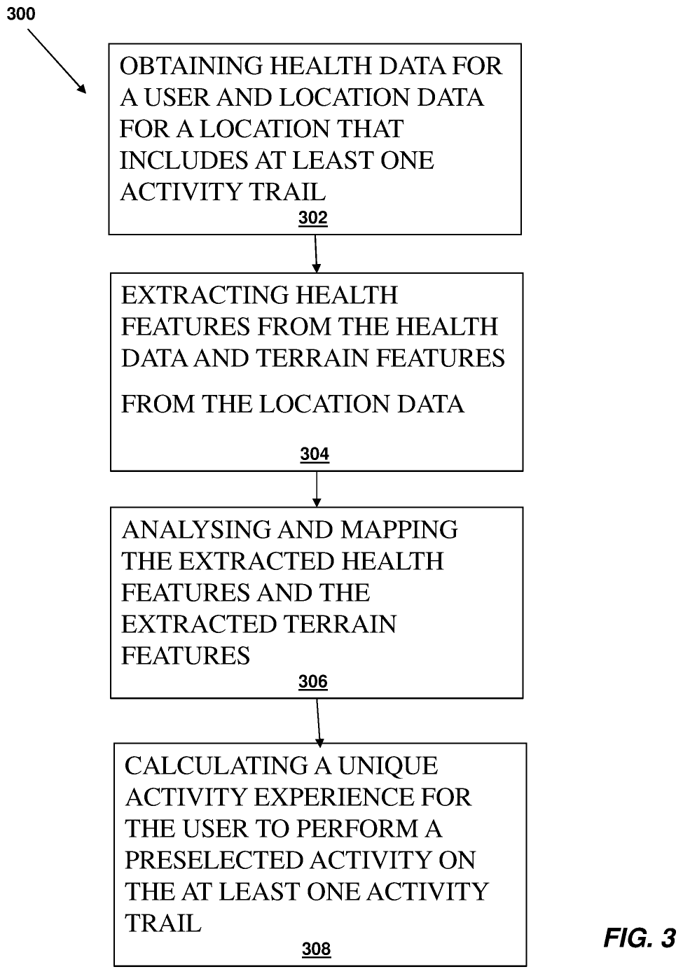

FIG. 3 illustrates a flowchart of unique hiking interaction via IoT data assimilation 300, in accordance with an exemplary embodiment of the present inventive concept.

The unique hiking interaction via IoT data assimilation 300 can include steps for:
Obtaining health data for a user and location data for a location that includes at least one activity trail (step 302);
Extracting health features from the health data and terrain features from the location data (step 304);
Analysing and mapping the extracted health features and the extracted terrain features, wherein the extracted health features include biometric measurements from at least one IoT device, and wherein the extracted terrain features include characteristics of the at least one activity trail (step 306); and
Calculating a unique activity experience for the user to perform a preselected activity on the at least one activity trail based at least in part on the analysed and mapped extracted health features and the extracted terrain features (step 308).

Based on the foregoing, a computer system, method, and computer program product have been disclosed. However, numerous modifications, additions, and substitutions can be made without deviating from the scope of the exemplary embodiments of the present inventive concept. Therefore, the exemplary embodiments of the present inventive concept have been disclosed by way of example and not by limitation.

What is claimed is:

1. A method of unique hiking interaction via IoT data assimilation, the method comprising:
obtaining health data for a user and location data for a location that includes at least one activity trail;
extracting health features from the health data and terrain features from the location data;
analysing and mapping the extracted health features and the extracted terrain features, wherein the extracted health features include biometric measurements from at least one IoT device, and wherein the extracted terrain features include characteristics of the at least one activity trail; and
calculating a unique activity experience for the user to perform a preselected activity on the at least one activity trail based at least in part on the analysed and mapped extracted health features and the extracted terrain features.

2. The method of claim 1, further comprising:
determining a user fitness level and an activity trail difficulty level based on the analysed extracted health features and the analysed extracted terrain features.

3. The method of claim 2, further comprising:
comparing the determined user fitness level to the determined activity trail difficulty level, wherein the calculated unique activity experience is based at least in part on the comparison of the determined user fitness level and the determined activity trail difficulty level.

4. The method of claim 2, further comprising:
selecting the at least one activity trail from among a plurality of activity trails included at the location by comparing the determined user fitness level and the determined activity trail difficulty level.

5. The method of claim 1, wherein the calculated unique activity experience includes an estimated personal completion time, route, progress benchmarks, predicted biometrics, and predicted positions of the user while performing the at least one preselected activity on the at least one activity trail.

6. The method of claim 1, wherein the calculated unique activity experience is displayed on a user interface and dynamically updated based on real-time biometrics, real-time progress, and real-time terrain features.

7. The method of claim 1, wherein the preselected activity is hiking, and wherein the calculated unique activity experience includes notifying the user of at least one of hazards, considerations, suggested routes, landmarks, and suggested equipment via a user interface.

8. A computer program product (CPP) for unique hiking interaction via IoT data assimilation, the CPP comprising:
one or more computer-readable storage media and program instructions stored on the one or more non-transitory computer-readable storage media capable of performing a method, the method comprising:
obtaining health data for a user and location data for a location that includes at least one activity trail;
extracting health features from the health data and terrain features from the location data;
analysing and mapping the extracted health features and the extracted terrain features, wherein the extracted health features include biometric measurements from at least one IoT device, and wherein the extracted terrain features include characteristics of the at least one activity trail; and
calculating a unique activity experience for the user to perform a preselected activity on the at least one activity trail based at least in part on the analysed and mapped extracted health features and the extracted terrain features.

9. The CPP of claim 8, further comprising:
determining a user fitness level and an activity trail difficulty level based on the analysed extracted health features and the analysed extracted terrain features.

10. The CPP of claim 9, further comprising:
comparing the determined user fitness level to the determined activity trail difficulty level, wherein the calculated unique activity experience is based at least in part on the comparison of the determined user fitness level and the determined activity trail difficulty level.

11. The CPP of claim 9, further comprising:
selecting the at least one activity trail from among a plurality of activity trails included at the location by comparing the determined user fitness level and the determined activity trail difficulty level.

12. The CPP of claim 8, wherein the calculated unique activity experience includes an estimated personal completion time, route, progress benchmarks, predicted biometrics, and predicted positions of the user while performing the at least one preselected activity on the at least one activity trail.

13. The CPP of claim 8, wherein the calculated unique activity experience is displayed on a user interface and dynamically updated based on real-time biometrics, real-time progress, and real-time terrain features.

14. The CPP of claim 8, wherein the preselected activity is hiking, and wherein the calculated unique activity experience includes notifying the user of at least one of hazards, considerations, suggested routes, landmarks, and suggested equipment via a user interface.

15. A computer system (CS) for unique hiking interaction via IoT data assimilation, the CS comprising:
one or more computer processors, one or more computer-readable storage media, and program instructions stored on the one or more of the computer-readable storage media for execution by at least one of the one or more processors capable of performing a method, the method comprising:
obtaining health data for a user and location data for a location that includes at least one activity trail;
extracting health features from the health data and terrain features from the location data;
analysing and mapping the extracted health features and the extracted terrain features, wherein the extracted health features include biometric measurements from at least one IoT device, and wherein the extracted terrain features include characteristics of the at least one activity trail; and
calculating a unique activity experience for the user to perform a preselected activity on the at least one activity trail based at least in part on the analysed and mapped extracted health features and the extracted terrain features.

16. The CS of claim 15, further comprising:
determining a user fitness level and an activity trail difficulty level based on the analysed extracted health features and the analysed extracted terrain features.

17. The CS of claim 16, further comprising:
comparing the determined user fitness level to the determined activity trail difficulty level, wherein the calculated unique activity experience is based at least in part on the comparison of the determined user fitness level and the determined activity trail difficulty level.

18. The CS of claim 16, further comprising:
selecting the at least one activity trail from among a plurality of activity trails included at the location by comparing the determined user fitness level and the determined activity trail difficulty level.

19. The CS of claim 15, wherein the calculated unique activity experience includes an estimated personal completion time, route, progress benchmarks, predicted biometrics, and predicted positions of the user while performing the at least one preselected activity on the at least one activity trail.

20. The CS of claim 15, wherein the calculated unique activity experience is displayed on a user interface and dynamically updated based on real-time biometrics, real-time progress, and real-time terrain features.
